(19) United States
(12) Patent Application Publication
(10) Pub. No.: US 2023/0313123 A1
(43) Pub. Date: Oct. 5, 2023

(54) MICROORGANISMS CAPABLE OF DECOMPOSING THERMOPLASTIC POLYURETHANE, METHOD FOR DECOMPOSING THERMOPLASTIC POLYURETHANE, AND METHOD FOR SELECTING MICROORGANISMS CAPABLE OF DECOMPOSING THERMOPLASTIC POLYURETHANE

(71) Applicants: NIHON PLAST CO., LTD., Fujinomiya-shi (JP); NATIONAL UNIVERSITY CORPORATION HOKKAIDO HIGHER EDUCATION AND RESEARCH SYSTEM, Obihiro-shi (JP)

(72) Inventors: Kazuyuki ENDO, Fujinomiya-shi (JP); Takuji OHWADA, Obihiro-shi (JP)

(21) Appl. No.: 18/128,822

(22) Filed: Mar. 30, 2023

(30) Foreign Application Priority Data
Mar. 30, 2022 (JP) 2022-055847

(51) Int. Cl. C12N 1/20; C08J 11/04; C12R 1/38; C12R 1/265

(52) U.S. Cl. CPC C12N 1/205; C08J 11/04; C12R 2001/38; C12R 2001/265; C08J 2375/04

(57) ABSTRACT

Microorganisms capable of effectively decompose hardly decomposable thermoplastic polyurethane, a method for decomposing thermoplastic polyurethane, and a method for selecting microorganisms capable of decomposing thermoplastic polyurethane are provided.

The method for selecting microorganisms capable of decomposing thermoplastic polyurethane includes a step of burying in soil a mixed sample of thermoplastic polyurethane having urea bond and polyurethane without urea bond and a step of collecting the microorganism adsorbing to the mixed sample after confirming the urethane decomposition by infrared spectroscopic analysis of the buried mixed sample.

### INCORPORATION BY REFERENCE

This application is based upon and claims the benefit of priority from the corresponding Japanese Patent Application No. 2022-055847 filed on Mar. 30, 2022, the entire contents of which are incorporated herein by reference.

### TECHNICAL FIELD

The present invention relates to microorganisms capable of decomposing thermoplastic polyurethane, a method for decomposing thermoplastic polyurethane, and a method for selecting microorganisms capable of decomposing thermoplastic polyurethane BACKGROUND ART

Polyurethane is a polymer having a urethane bond and is also called a urethane resin. Polyurethane is gradually decomposed under the influence of hydrolysis by moisture, ultraviolet rays, heat, microorganisms, and the like, and easily degrade its physical properties. However, polyurethane is usually modified to mitigate decomposition by improving its structure that is less susceptible to hydrolysis and microorganisms.

Polyurethane, which has cross-linking structure, cannot be used as a recycling material by melting in the same way as thermoplastic resins, and is disposed of in landfills, causing environmental problems. Currently, in order to avoid this problem, studies on using polyurethane as a recycling material by exploiting decomposition action of microorganism, which is environmentally friendly, and biodegradation of polyurethane are underway. However, it takes a lot of time and efforts to find microorganisms effective for such purposes, and few examples have led to practical applications.

The methods described in JP-A-2010-220610 (PTL1) and JP-A-2015-128407 (PTL2), for example, are known to decompose polyurethane utilizing decomposition by microorganism. Patent Literature 1 describes using C13a strain (actinomycetes) belonging to a genus Streptomyces as a microorganism having urethane decomposing function. Furthermore, Patent Literature 2 discloses a urethane decomposing method having a step of pretreating a urethane-containing material to be treated using an unsaturated fatty acid such as oleic acid, and a step of making a microorganism exhibiting urethane decomposing function (such as the said C13a belonging to a genus Streptomyces) to act on the material treated using the unsaturated fatty acid.

### PATENT LITERATURE

[PTL 1] JP-A-2010-220610
[PTL 2] JP-A-2015-128407

### SUMMARY OF INVENTION

### Technical Problem

In accordance with the methods described in PTL 1 and PTL 2 as above, soft polyurethane form can be effectively decomposed using the decomposition action of microorganisms. Therefore, the inventors of this application tried to decompose thermoplastic polyurethane (TPU) based on the methods described in PTL 1 and PTL 2. However, no changes in surface condition of thermoplastic polyurethane, which generally decomposes with difficulty, were found, i.e., no progress of thermoplastic polyurethane decomposition was observed. On the other hand, as a comparison, polyether-base urethane foam was decomposed in a similar method, and it was found that the weight of the polyether-base urethane foam reduced by a little less than 4% in weight. Furthermore, formation of micropores in independent foam cells was confirmed, suggesting that decomposition progressed.

The purpose of the present invention is to provide microorganisms capable of effectively decompose hardly decomposable thermoplastic polyurethane, a method for decomposing thermoplastic polyurethane, and a method for selecting microorganisms capable of decomposing thermoplastic polyurethane.

### Solution to Problem

The microorganisms in accordance with one embodiment of the present invention belong to a genus Pseudomonas and a genus Sinomonas that are capable of decomposing thermoplastic polyurethane containing urea bond.

The method for decomposing thermoplastic polyurethane in accordance with one embodiment of the present invention includes a step of making the microorganism microorganisms in relation to one embodiment of the present invention act on the thermoplastic polyurethane containing urea bond.

The method for selecting microorganisms capable of decomposing thermoplastic polyurethane in accordance with one embodiment of the present invention includes:
a step of burying in soil a mixed sample of thermoplastic polyurethane containing urea bond and polyurethane not containing urea bond and
a step of collecting the microorganism adsorbing to the mixed sample after confirming the urethane decomposition by infrared spectroscopic analysis of the buried mixed sample.

### Advantageous Effects of Invention

In accordance with the present invention, microorganisms capable of effectively decompose hardly decomposable thermoplastic polyurethane, a method for decomposing thermoplastic polyurethane, and a method for selecting microorganisms capable of decomposing thermoplastic polyurethane are provided.

### DESCRIPTION OF DRAWINGS

FIG. 1 shows infrared spectroscopic spectra for mixed samples 14 months after the mixed samples were buried in accordance with examples of the present application.

FIG. 2 shows infrared spectroscopic spectra for mixed samples 17 months after the mixed samples were buried in accordance with examples of the present application.

FIG. 3 shows weight reduction ratios of thermoplastic polyurethane in the examples of the present application wherein novel decomposing bacteria in accordance with the present invention were made to act on thermoplastic polyurethane without pretreating.

FIG. 4 shows weight reduction ratios of thermoplastic urethane wherein the microorganisms were made to act on thermoplastic polyurethane after plasma treatment in the examples of the present application.

FIG. 5 shows weight reduction ratios of thermoplastic polyurethane wherein the novel decomposing bacteria in accordance with the present invention were made to act on the thermoplastic polyurethane after plasma treatment and treatment with oleic acid in the examples of the present application.

FIG. 6 shows the comparison of the pore numbers in the thermoplastic polyurethane after the novel decomposing bacteria in accordance with the present invention were made to act on thermoplastic polyurethane in the examples of the present application.

FIG. 7 shows SEM images of the surface of the thermoplastic polyurethane after the novel decomposing bacteria in accordance with the present invention were made to act on the thermoplastic polyurethane.

FIG. 8 shows the results of infrared spectroscopic analyses of the thermoplastic polyurethane after the novel decomposing bacteria in accordance with the present invention were made to act on the thermoplastic polyurethane.

FIG. 9 shows the melting properties of the thermoplastic polyurethane after the novel decomposing bacteria in accordance with the present invention were made to act on the thermoplastic polyurethane.

FIG. 10 is a photograph showing the melting properties of the thermoplastic polyurethane after ES2231 strain, one of the novel decomposing bacteria in accordance with the present invention, was made to act on the thermoplastic polyurethane.

FIG. 11 is a photograph showing ash residues after the thermoplastic polyurethane, on which ES2231 strain, one of the novel decomposing bacteria in accordance with the present invention, was made to act, is heated to 700° C.

FIG. 12 is a photograph showing the melting properties of the molded product of the thermoplastic polyurethane.

FIG. 13 is a photograph showing the melting properties of the powdery raw material of the thermoplastic polyurethane.

FIG. 14 is a schematic showing the structure of the hardly decomposable thermoplastic polyurethane.

### DESCRIPTION OF EMBODIMENTS

The inventors of the present application tried to find the reason that decomposition of hardly decomposable thermoplastic polyurethane does not progress even if unsaturated fatty acid or microorganism is made to act on the hardly decomposable thermoplastic polyurethane. As a result, the inventors of the present application reached a conclusion that the reason that the decomposition of thermoplastic polyurethane hardly progress by the reaction of the thermoplastic polyurethane and unsaturated fatty acid or microorganism is due to the molecular structure of the thermoplastic polyurethane.

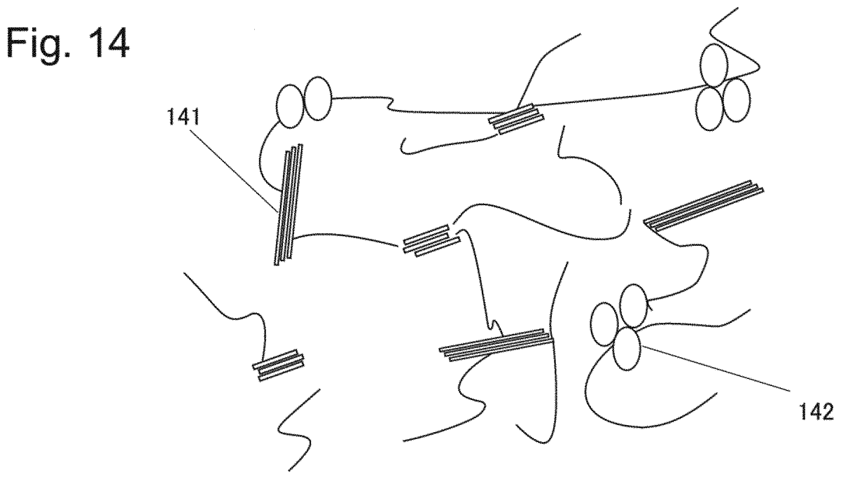

As shown in FIG. 14, hardly decomposable thermoplastic polyurethane has a structure of the hard segment and soft segment being phase-separated (sea-island structure). Polyurethane having urea bonds is used for the material of hard segment, and polyether-base polyurethane is used for the material of soft segment.

A urea bond has a symmetrical conjugated structure with a carbonyl group (═CO) in between as represented by formula (1) below, and has a property of being difficult to be decomposed because bonding force between molecules becomes stronger (see formula (3) below) by polarization as shown in formula (2) below. Furthermore, a urea bond has a property of being difficult to be decomposed against external attacks since valance electrons move causing relaxation.

Furthermore, since the polyether-based polyurethane in the soft segment contains highly cohesive polyester groups, the end groups hydrogen-bond with the urethane groups during the melt-solidification process to form partial cross-links, resulting in a stronger, more hardly decomposable structure. Thermoplastic polyurethane is known to cause marine pollution as microplastics. Therefore, if technology for recycling thermoplastic polyurethane as material is established, total amount of plastics can be reduced, leading to the alleviation of marine pollution.

Thermoplastic polyurethane, which has a hardly decomposable and stable property as described above, is slush molded to be used for cover materials of instrument panels for automobiles. In particular, such cover materials, having a good touch and feel with a high durability, have been widely adopted for luxury cars.

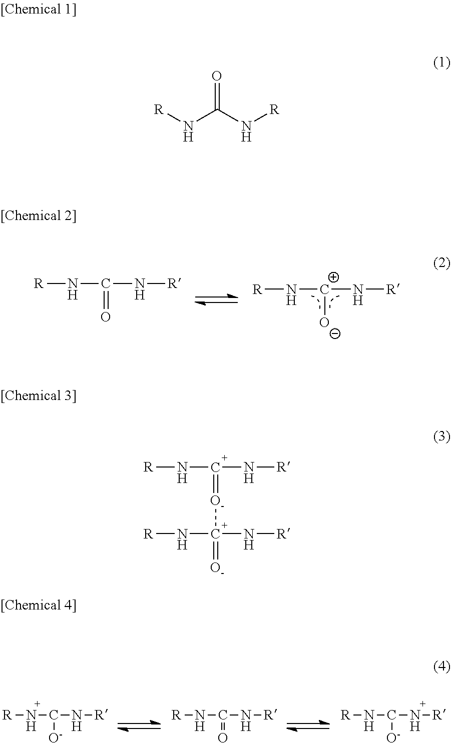

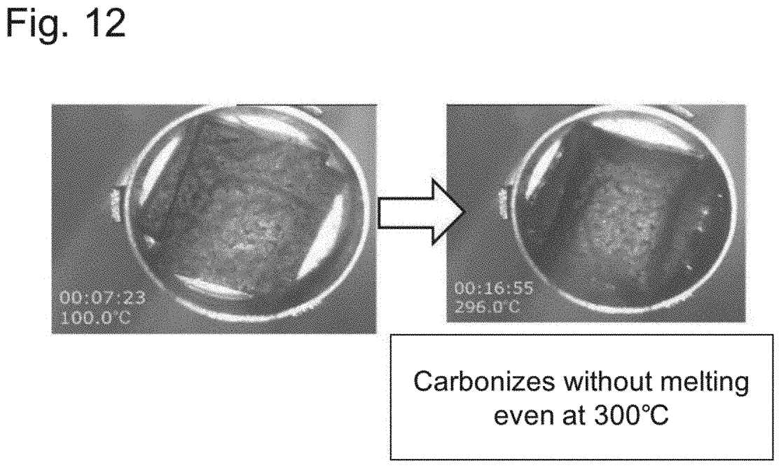

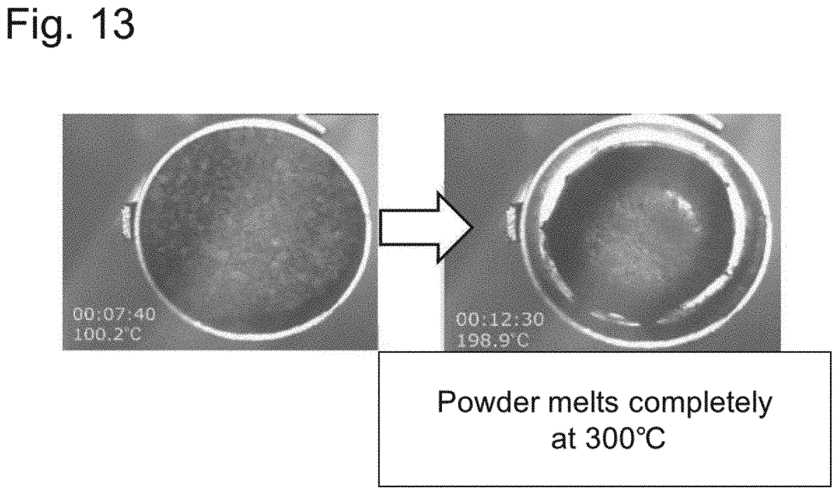

Powder material of thermoplastic polyurethane having 100 μm in size is molded by powder slush molding. The powder material is cast into a mold and is uniformly melted by heat and formed into sheets (see FIG. 13). Since cross-linking reaction takes place at the end portions of thermoplastic polyurethane during the process of melt-solidification as stated above, molded thermoplastic polyurethane sheets do not melt uniformly and decompose by carbonization even if heated again to about 40° C. (see FIG. 12) Therefore, waste materials of thermoplastic polyurethane cannot be recycled as thermoplastic resin by melting, thus only the thermal energy generated during incineration of them are currently utilized.

In order to recycle the waste materials of thermoplastic polyurethane, the inventors of this application, after a number of diligent studies on a method for selecting microorganism strain highly capable of decomposing hardly decomposable thermoplastic polyurethane, found that a method for selecting microorganisms capable of decomposing thermoplastic polyurethane in accordance with embodiments of the present invention as follows is effective.

Method for Selecting Microorganisms Capable of Decomposing Thermoplastic Polyurethane

The method for selecting microorganisms capable of decomposing thermoplastic polyurethane in accordance with embodiments of the present invention includes a step of burying in soil a mixed sample of thermoplastic polyurethane having urea bond and polyurethane without urea bond and a step of collecting the microorganism adsorbing to the mixed sample after confirming the urethane decomposition by infrared spectroscopic analysis of the buried mixed sample. Each step is described in detail below.

Step of Burying a Mixed Sample in Soil

Bacteria highly capable of decomposing hardly decomposable thermoplastic polyurethane need to be selected by collecting as many possible bacteria as possible. As stated above, since hardly decomposable thermoplastic polyurethane includes urea bonds that are hardly decomposable, novel decomposing bacteria that have a high adsorption to urea bonds need to be selected. However, even if hardly decomposable thermoplastic polyurethane is buried in soil as specimen, the number of bacteria to accumulate is limited, thus effective bacteria can not be selected.

Therefore, in the method for selecting microorganisms capable of decomposing thermoplastic polyurethane in accordance with one embodiment of the present invention, mixed samples are provided by mixing easily decomposable general-purpose polyurethane containing no urea bond and hardly decomposable thermoplastic polyurethane and the mixed samples are used as specimen and buried in soil. This creates an environment conducive to attracting bacteria that is easy to adsorb to urea bonds. There are no particular limitations on thermoplastic polyurethane containing urea bond and polyurethane not containing urea bond, respectively. Waste materials in the shape of a sheet can be used, and the mixed samples are prepared by adjusting the size thereof as appropriate and mixing them. It is preferable to attract as many bacteria as possible to the surface of the mixed samples in soil, and select bacteria capable of decomposing thermoplastic polyurethane containing urea bond which is hardly decomposable. For this purpose, the mixed samples preferably contain more polyurethane not containing urea bond. The mixing ratio of polyurethane not containing urea bond and thermoplastic polyurethane containing urea bond is not particularly limited, however, in light of the above, they can be mixed at a ratio of about 70:30 by mass, for example.

Step of Collecting Microorganism

Next, the mixed samples buried in soil are periodically collected for analysis to confirm the progress of urethane decomposition. Urethane decomposition can be confirmed by looking at the changes in absorption peak at 1100 cm⁻¹ in the spectra of infrared spectroscopic analysis, which indicates the presence of ether bond (C—O—C). Reduction in absorption peak at 1100 cm⁻¹ in the spectrum waveform suggests the number of ether bonds is reduced, suggesting a sign of urethane decomposition.

When a sign of urethane decomposition is confirmed for the mixed sample, bacteria adsorbing to the mixed sample are collected, and are selected as a candidate strain of urethane-decomposing bacteria (primary selection).

Step of Culturing Microorganism

Bacteria capable of decomposing hardly decomposable polyurethane containing urea bond can be included in the bacteria collected as described above. Therefore, a further selection of bacteria (secondary selection) is conducted to determine their decomposition ability.

Specifically, bacteria with a high growth rate are further selected by shaking-culturing the bacteria collected above using an inorganic salt medium containing simple reference material as carbon and nitrogen sources with urea bond that is the target for decomposition. For the simple reference material with urea bond, for example, 1,3-dimethylurea (DMU) (see (5) below) or 1,3-diethylurea (DEU) (see (6) below) can be used.

Since DMU and DEU, have simple structures on which bacteria can act without the influence of other functional groups, microorganisms capable of decomposing thermoplastic polyurethane containing urea bond can be selected more easily.

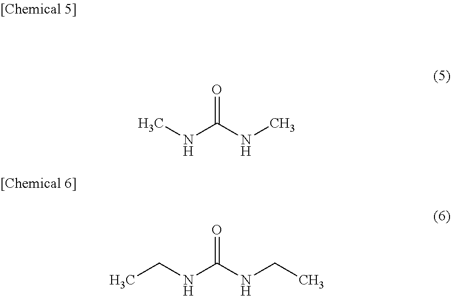

Since bacteria proliferated in the inorganic salt medium containing DMU, and/or, DEU grow by decomposing urea bond and using it as carbon and nitrogen sources, they are microorganisms capable of decomposing polyurethane containing urea bond.

Excellent bacteria strains having a higher urethane decomposing ability may be separated from the bacteria selected in the above manner by checking the state of adsorption of the bacteria to thermoplastic polyurethane and by setting quantitative criteria for urethane decomposing ability. For example, bacteria strains having the highest urethane decomposing ability may be separated using the following method.

Analysis of Infrared Spectroscopy Novel decomposing bacteria can be evaluated for urethane decomposing ability by infrared spectroscopic analysis for the thermoplastic polyurethane on which the novel decomposing bacteria was made to act. The peak at 740 cm⁻¹ of the spectrum in infrared spectroscopic analysis is characteristic of primary amine formed when the urea bond is decomposed.

When resin containing urea bond goes through hydrolysis, it is presumed that a water molecule acts on C—N bond in secondary amine as shown in chemical formula (7), forming a primary amine of R—NH₂. When urea bond is broken, the peak in infrared spectra appears for a primary amine, while the peak does not appear for a secondary amine. Thus, the progress of urethane decomposition can be estimated by measuring changes in the peak at around 740 cm⁻¹ in the spectra.

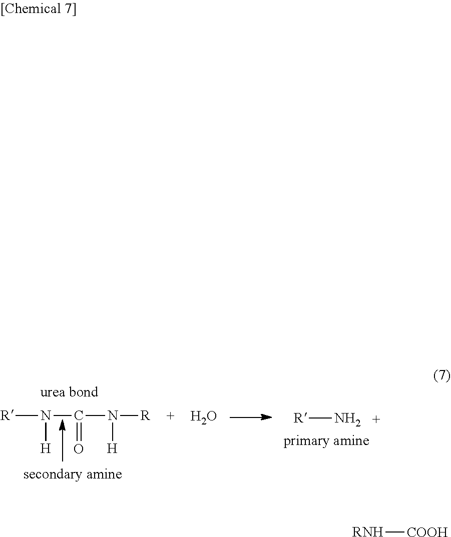

As described above, correlation with the weight loss of thermoplastic polyurethane can be obtained by focusing on the peak ratios at around 740 cm⁻¹ in the spectra in infrared spectroscopic analysis. Thus, excellent bacteria strains can be selected by determining correctly the urethane decomposition by the novel decomposing bacteria in light of the molecular structural changes in thermoplastic polyurethane.

SEM Observation and Pore Number Counting by ImageJ

SEM Observation of the Surface of Thermoplastic Polyurethane

The adsorption of the novel decomposing bacteria can be evaluated by observing 2000-power photos of SEM image of the surface of the thermoplastic polyurethane on which the novel decomposing bacteria were made to act.

Measurement of the Pore Number

The number of pores of the thermoplastic polyurethane treated with the novel decomposing bacteria is measured using ImageJ, an image processing software, in particle analysis mode. Quantitative analyses of the state of the pores (the number of pores, total pore area, ratio of total pore area, etc.) emerging on the surface of the thermoplastic polyurethane after the decomposition by the novel decomposing bacteria allow to grasp the characteristics of the novel decomposing bacteria for urethane decomposing ability, which makes possible the selection of bacteria suitable for recycling thermoplastic polyurethane as material. Measuring conditions include, for example, setting the threshold to between 0 and pore recognizable value (normally 60-100) and the pore size to between 10 and ∞ pixel (1 μm=7.97 pixel).

Evaluation of Melting Properties by Thermal Analysis

Thermal analyses of the thermoplastic polyurethane treated with the novel decomposing bacteria can be made for softening start temperature, melting end temperature, boiling point and decomposition start temperature to evaluate the advantageous properties when recycled as thermoplastic resin, in which a thermal analysis device (TG-DTA) can be used for this purpose.

Microorganism Capable of Decomposing Thermoplastic Polyurethane

Microorganisms capable of decomposing thermoplastic polyurethane in accordance with one embodiment of the present invention belong to genus Pseudomonas genus and a genus Sinomonas that are capable of decomposing thermoplastic polyurethane containing urea bond. More specifically, the microorganism in accordance with one embodiment of the present invention is preferably Pseudomonas hibiscicola MS4102 strain which was deposited with International Patent Organism Depository, National Institute of Technology and Evaluation on Feb. 24, 2022 by Accession No. NITE P-03612 or Sinomonas atrocyanea ES2231 strain which was deposited with International Patent Organism Depository, National Institute of Technology and Evaluation on Feb. 24, 2022 by Accession No. NITE P-03613.

These microorganisms are selected from the soil by the method for selecting microorganisms capable of decomposing thermoplastic polyurethane in accordance with one embodiment of the present invention as described below.

Method for Decomposing Thermoplastic Polyurethane

The method for decomposing thermoplastic polyurethane in accordance with one embodiment of the present invention includes a step of making the microorganisms capable of decomposing thermoplastic polyurethane in relation to one embodiment of the present invention as described above act on the thermoplastic polyurethane containing urea bond.

Methods of making the microorganism act on thermoplastic polyurethane include, for example, adding the thermoplastic polyurethane to the culture medium for the microorganisms.

In case that the thermoplastic polyurethane is in the form of a sheet, it is preferably cut into small pieces of about 1 cm by 1 cm before treatment. This allows an easier recycling of the thermoplastic polyurethane after treatment with the microorganisms. Examples of recycling methods for the thermoplastic polyurethane after treatment with the microorganisms include melting the thermoplastic polyurethane with a biaxial kneading machine and forming into injection molding pellets.

The microorganisms capable of decomposing thermoplastic polyurethane, the method for decomposing thermoplastic polyurethane and the method for selecting microorganisms capable of decomposing thermoplastic polyurethane include the following aspects.
(1) The microorganisms belong to a genus Pseudomonas and a genus Sinomonas and that are capable of decomposing thermoplastic polyurethane containing urea bond.
(2) The microorganism according to (1) above is Pseudomonas hibiscicola MS4102 strain specified by Accession No. NITE P-03612.
(3) The microorganism according to (1) above is Sinomonas atrocyanea ES2231 strain specified by Accession No. NITE P-03613.
(4) A method for decomposing thermoplastic polyurethane that includes a step of making the microorganism according to (1) to (3) above act on the thermoplastic polyurethane containing urea bond.
   a step of burying in soil a mixed sample of thermoplastic polyurethane containing urea bond and polyurethane not containing urea bond and
   a step of collecting the microorganism adsorbing to the mixed sample after confirming the urethane decomposition by analysis of the buried mixed sample.
(5) A method for selecting microorganisms capable of decomposing thermoplastic polyurethane including
(6) The method for selecting microorganisms capable of decomposing thermoplastic polyurethane according to (5) above, wherein the method includes a step of culturing the said microorganism in an inorganic salt medium containing 1, 3-dimethylurea, 1, 3-diethylurea or 1, 3-dimethylurea and 1, 3-diethylurea.

### EXAMPLES

The present invention is described in more detail below with reference to the examples. The present invention, however, is not limited by these examples.

### Example

### Primary Selection

First, mixed samples are prepared and buried in soil for the primary selection. A mixed sample was prepared by mixing general-purpose polyurethane not containing urea bond and hardly decomposable thermoplastic polyurethane containing urea bond are mixed at a ratio of about 70:30 by mass. Furthermore, the mixed samples in the form of a sheet of about 1 cm by 1 cm were used.

The mixed samples buried in soil are periodically collected for spectrum waveform analysis of infrared spectroscopy to confirm urethane decomposition.

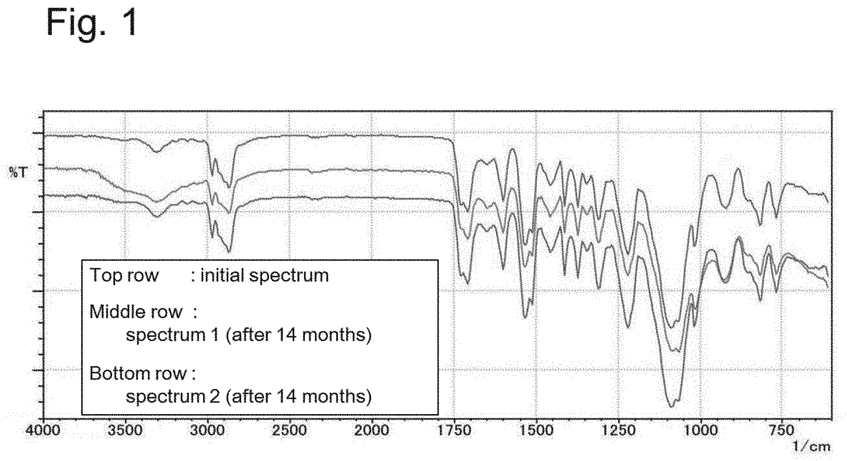

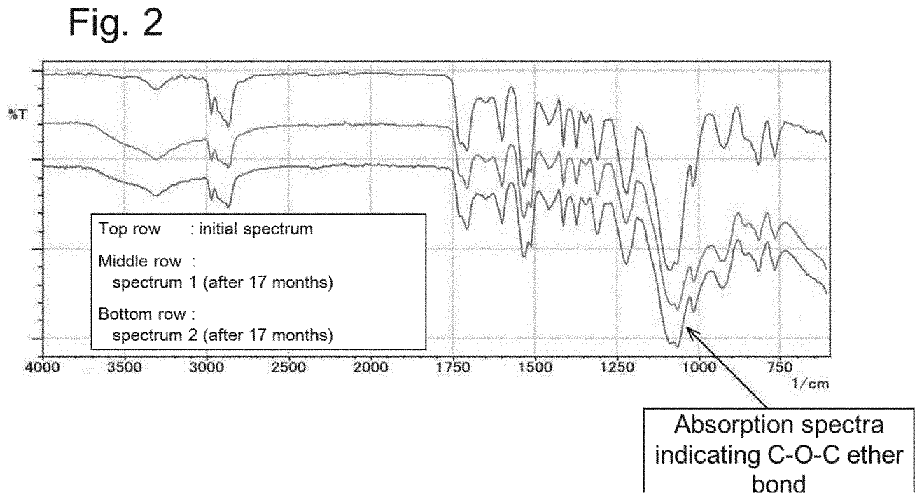

As a result, no changes in spectrum waveform were observed for the mixed samples until 14 months after they are buried in soil as shown in FIG. 1. On the other hand, a reduction in absorption peak at 1100 cm⁻¹ for ether bond (C—O—C) was observed for the mixed samples 17 months after being bury in soil as shown in FIG. 2, suggesting that decomposition was taking place. Thus, as the progress of urethane decomposition by soil bacteria is suggested, bacteria adsorbing to the mixed sample were sampled for searching for the novel decomposing bacteria. It should be noted that “Spectrum 1” specified in the middle row of FIGS. 1 and 2 is the result measured from the surface of the mixed sample, while “Spectrum 2” specified in the bottom row of the same figures is the result measured from the back side of the mixed sample.

Secondary Selection

In the secondary selection, the microorganism collected by the primary selection as above was cultured in an inorganic salt medium containing 1, 3-dimethylurea (DMU) or 1,3-diethylurea (DEU). In this way, bacteria with a high growth rate that can grow by using DMU or DEU as carbon and nitrogen sources can be selected from the microorganisms selected in the primary selection.

As a result of culturing the bacteria using media containing DMU, 36 separated bacteria (DMU assimilating bacteria) are selected. Next, the bacteria were narrowed down to 11 useful strains as shown in table 1 by evaluating each growth rate of the separated bacteria. In addition, the bacteria were further narrowed down to 5 strains by evaluating the growth rate of the bacteria on the media containing DEU as well as the growth rate of the bacteria.

TABLE 1

| | bacterial growth ability | | | |
|---|---|---|---|---|
| bacterial | R2A | DMU | DEU | configuration and shape of |
| strain | medium | medium | medium | colony |
| MS1411 | + | +++ | − | unclear (pale overall, partially |
| | | | | pink) |
| MS3232 | +++ | +++ | − | unclear (white and nebular) |
| MS3234 | +++ | +++ | − | small colony (white) |
| MS3244 | +++ | ++ | − | small colony (white) |
| MS3333 | ++++ | ++ | + | small colony (light yellow) |
| MS3344 | +++ | +++ | − | unclear outline (pale white or |
| | | | | transparent) |
| MS4101 | +++ | ++ | −+ | small colony (light yellow) |
| MS4102 | ++++ | +++ | ++ | small colony (light yellow) |
| MS4111 | ++++ | +++ | + | unclear outline (light yellow, |
| | | | | watery) |
| MS4303 | ++++ | +++ | ++ | small colony (light yellow) |
| MS4304 | ++++ | ++ | +++ | small colony (white) |

R2A medium: standard medium
DMU medium: inorganic salt medium containing DMU
DEU medium: inorganic salt medium containing DEU

Commercially available R2A Agar (BD Difco™) or R2A Broth DAIGO (FUJIFILM Wako Pure Chemical Corporation) was used for R2A medium.

The bacteria were smeared on agar medium and cultured, and the relative evaluation of the bacteria growth after culturing was made. Evaluation of the bacterial growth ability appears in Table 1. The evaluation criteria are as follows:
+: colony formation after the smeared bacteria partially proliferate
++: colony formation after about half of the smeared bacteria proliferate
+++: colony formation after all of the smeared bacteria proliferate
++++: colony formation after all of the smeared bacteria proliferate at a high growth rate
−: no colony formation without proliferation of the smeared bacteria
−+: unclear colony formation and proliferation of the smeared bacteria (apparent colony formation after the smeared bacteria partially proliferate, but no clear colony formation and proliferation of the smeared bacteria)

Furthermore, as a result of culturing the bacteria using media containing DEU, 44 separated bacteria (DEU assimilating bacteria) are selected. Next, the bacteria were narrowed down to 4 useful strains as shown in table 2 by evaluating each growth rate of the separated bacteria on the R2A medium and DEU medium.

TABLE 2

| | bacterial growth ability | | |
|---|---|---|---|
| bacterial | R2A | DEU | |
| strain | medium | medium | configuration and shape of colony |
| ES1243 | +++ | + | small colony (light yellow) |
| ES1247 | ++ | −+ | small colony (white) |
| ES1248 | + | −+ | colony having an unclear boundary |
| | | | with the media |
| ES2231 | +++ | ++ | colony having a clear boundary with |
| | | | the media (light yellow) |
| ES2241 | ++ | ++ | small colony (pale white) |
| ES2242 | ++ | ++ | colony having a clear boundary with |
| | | | the media (light yellow) |
| ES2243 | ++ | ++ | small colony (pale white) |

It is noted that the evaluation of the bacterial growth ability was conducted in the same way as in the bacterial growth ability shown in table 1.

By evaluating the bacterial growth ability as described above, 9 excellent bacteria strains (5 DMU assimilating bacteria and 4 DEU assimilating bacteria) are selected. Thus, searching steps for the identification of the selected bacteria strains can be greatly reduced by narrowing down the useful bacteria strains by evaluation of their growth ability. This method allows for an effective consideration for practical applications because such information as the properties and hazardousness of the bacteria can be swiftly grasped.

TABLE 3

| bacterial strain | most closely related strain |
|---|---|
| MS3333 | Pseudomonas aeruginosa |
| MS4102 | Pseudomonas hibiscicola |
| MS4111 | Brevundimonas olei |
| MS4303 | Pseudomonas aeruginosa |
| MS4304 | Stenotrophomonas maltophilia |
| ES1243 | Rhizobium nepotum |
| ES1247 | Pseudomonas nitroreducens |
| ES2231 | Sinomonas atrocyanea |
| ES2242 | Sinomonas flava |

The used databases are as follows:
Ribosomal Database Project (RDP)
National Center for Biotechnology Information (NCBI)
DNA Data Bank of Japan (DDBJ)

Two bacterial strains as below are selected as decomposing bacteria having a high adsorption and growth rate from the 9 decomposing bacteria that were narrowed down in the secondary selection.

MS4102 Strain (Pseudomonas hibiscicola)
Domain: Bacteria
Phylum: Proteobacteria
Class: Gammaproteobacteria
Order: Pseudomonadales
Family: Pseudomonadaceae
Genus: Pseudomonas

MS4111 Strain (Brevundimonas olei)
Domain: Bacteria
Phylum: Proteobacteria
Class: Alphaproteobacteria
Order: Caulobacterales
Family: Caulobacteraceae
Genus: Brevundimonas

ES1243 Strain (Rhizobium nepotum)
Domain: Bacteria
Phylum: Proteobacteria
Class: Alphaproteobacteria
Order: Rhizobiales
Family: Rhizobiaceae
Genus: Rhizobium

ES2231 Strain (Sinomonas atrocyanea)
Domain: Bacteria
Phylum: Actinobacteria
Class: Actinobacteria
Order: Micrococcales
Family: Micrococcaceae
Genus: Sinomonas

Evaluation of Decomposition of Thermoplastic Polyurethane

The 4 separated bacteria strains were made to act on thermoplastic polyurethane to evaluate their urethane decomposing ability in a method described below.

### Preparation

molding of thermoplastic polyurethane (1 cm×1 cm×0.5 mm (0.5 mm in thickness))
weight measurement of thermoplastic polyurethane (a: weight just before culturing)

### Culturing

preculturing of the separated bacteria
collection of precultured bacteria and washing of thermoplastic polyurethane
main culturing on the medium with added thermoplastic polyurethane (30° C., three weeks, 100 rpm).

### Measurement

weight measurement of thermoplastic polyurethane ((3: weight measured after culturing)
SEM observation of thermoplastic polyurethane
Image analysis of thermoplastic polyurethane using ImageJ (counting of pore numbers)

Weight reduction ratio of thermoplastic polyurethane measured before and after the separated bacteria are made to act was calculated respectively based on the following formula.

weight reduction ratio (%)=(α−β)/β×100 (5)

The separated bacteria were cultured with a pH level of 7 at a liquid temperature of 30° C., where a high growth rate of bacteria is expected. The composition of the media used for culturing (inorganic salt liquid media) is shown on Table 4.

TABLE 4
composition of inorganic salt liquid media (original concentration)

| | | |
|---|---|---|
| DW | 1 | L |
| KH₂PO₄ | 2 | g |
| K₂HPO₄ | 7 | g |
| MgSO₄•7H₂O | 100 | mg |
| ZnSO₄•7H₂O | 1 | mg |
| FeSO₄•7H₂O | 10 | mg |
| MnSO₄•7H₂O | 2 | mg |
| CuSO₄•5H₂O | 8.75 | μg |

* Nakajima-Kambe, Toshiaki et al.

Furthermore, before the separated bacteria were made to act on thermoplastic polyurethane, the effect of pretreatment was evaluated. The pretreatment methods to enhance the decomposition of thermoplastic polyurethane include a pretreatment with oleic acid disclosed in Japanese Patent No. 6489542 as a chemical treatment and a plasma pretreatment disclosed in JP2021-161338 as a physical treatment. For each one of the cases, the samples with the pretreatment are compared with the ones without pretreatment.

The conditions for the plasma treatment are as follows:
Plasma emission irradiation conditions
Pressure inside the chamber: 40 Pa
RF power source: 100 V
Bias voltage: 600 V
Sample height: 23 mm
Source gas: atmosphere
Plasma irradiation time: 120 seconds

Conditions for treatment of oleic acid is as follows:

Oleic acid (Wako extra pure grade of Wako Pure Chemical Industries, Ltd.) with a concentration of 10% (w/w) in concentration was prepared by diluting it with 99% ethanol was put in a 100 ml conical flask

About 4 to 5 g of thermoplastic polyurethane was put in the said 100 ml conical, is completely immersed, and treated at normal temperature for 1 hour by covering the 100 ml conical flask with aluminum foil.

After the elapse of the treatment time, the inside of the Erlenmeyer flask was washed using tap water and distilled water, distilled water was thereafter further added to the conical flask, and the Erlenmeyer flask was washed with ultrasound. The thermoplastic polyurethane was taken out from the Erlenmeyer flask and then further washed using distilled water. Then, the thermoplastic polyurethane was sufficiently dried (overnight) at 40° C., and was then subjected to a sterilization treatment under the conditions of 121° C. for 20 minutes.

Metal ions have catalytic function to promote autoxidation reactions of polymers for plastics, which is called redox reaction. A copper ion is especially sensitive to the reaction among other metal ions (copper damage). In particular, redox reaction easily progresses if carboxyl group (ROOH) or carbonyl group (OCO) is present inside.

Therefore, decomposition enhancement by redox reaction of metal ions when the separated bacteria were made to act on thermoplastic polyurethane was evaluated. Specifically, comparisons were made by making the concentration of Cu ion and Fe ion in the media for culturing the separate bacteria 10 times, respectively.

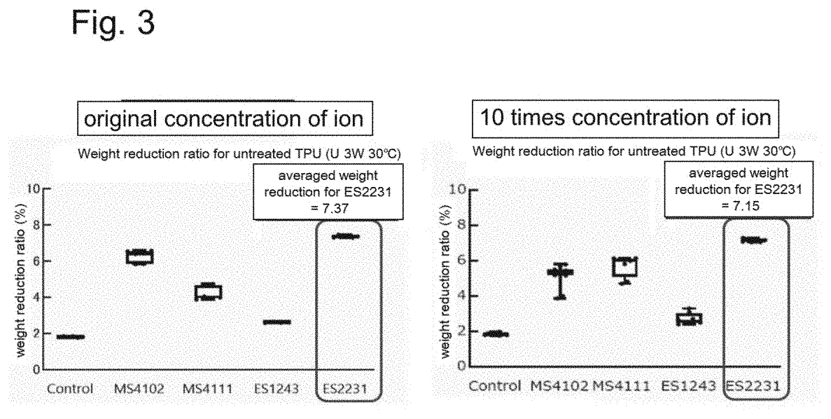

FIG. 3 shows the weight reduction ratios when the separate bacteria were made to act on the thermoplastic polyurethane without pretreatment (untreated TPU). The graph on the left in FIG. 3 is for original concentration of Cu ion and Fe ions in the media for culturing the separate bacteria, while the graph on the right in FIG. 3 is for Cu ion and Fe ions 10 times the concentration of the original in the media for culturing the separate bacteria.

As FIG. 3 shows, ES2231 strain, a novel decomposing bacterium, has over 7% weight reduction ratio, indicating that it has a high decomposing ability even without pretreatment for thermoplastic polyurethane that is not decomposed by conventional C13a actinomycete.

As for the enhancement of decomposition by redox reaction of metal ions, no sign of decomposition enhancement was observed when the concentration of Cu ion and Fe ion in the media for culturing the separate bacteria is 10 times.

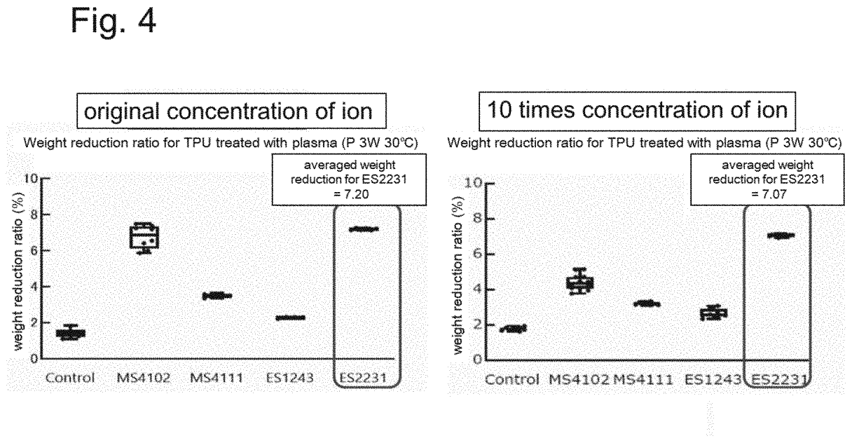

FIG. 4 shows the weight reduction ratios when the separate bacteria were made to act on the thermoplastic polyurethane after plasma treatment (plasma-treated TPU). The graph on the left in FIG. 4 is for original concentration of Cu ion and Fe ions in the media for culturing the separate bacteria, while the graph on the right in FIG. 3 is for Cu ion and Fe ions 10 times the concentration of the original in the media for culturing the separate bacteria.

As FIG. 4 shows, ES2231 strain, similar to the case where the pretreatment was not applied, a novel decomposing bacterium has the highest decomposing ability. It should be noted that ES2231 strain in this case has about 7% weight reduction ratio, which is slightly lower than the one without pretreatment, and no sign of decomposition enhancement due to ion concentration changes was observed.

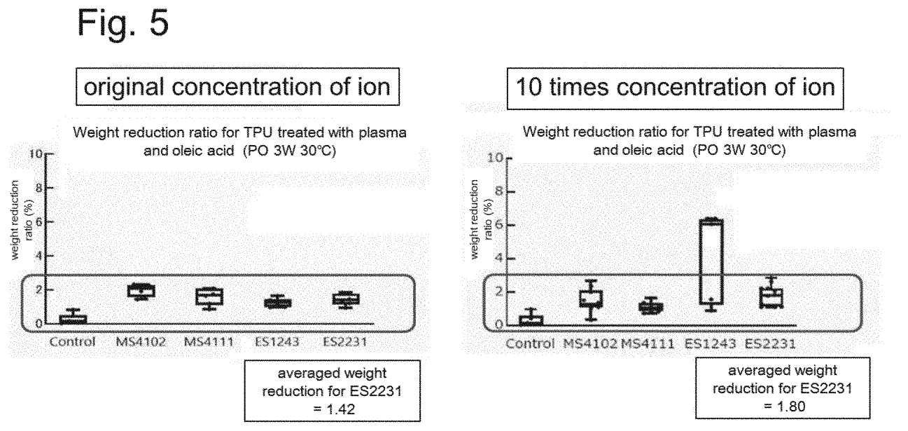

FIG. 5 shows the weight reduction ratios when the separate bacteria were made to act on the thermoplastic polyurethane after plasma treatment and oleic acid treatment (plasma-oleic acid-treated TPU). The graph on the left in FIG. 5 is for original concentration of Cu ion and Fe ions in the media for culturing the separate bacteria, while the graph on the right in FIG. 3 is for Cu ion and Fe ions 10 times the concentration of the original in the media for culturing the separate bacteria.

As FIG. 5 shows, the weight reduction ratio decreases by oleic acid treatment, meaning it inhibits the decomposing ability of the novel decomposing bacteria, and no sign of decomposition enhancement due to Ion concentration changes was observed.

Pore Number Measurement

Next, the pore numbers in the thermoplastic polyurethane after the separated bacteria were made to act on the thermoplastic polyurethane was measured. Quantitative analyses of the state of the pores (the number of pores, total pore area, ratio of total pore area, etc.) emerging on the surface of the thermoplastic polyurethane after the decomposition by the novel decomposing bacteria allow to grasp the characteristics of the novel decomposing bacteria for urethane decomposing ability, which makes possible the selection of bacteria suitable for recycling thermoplastic polyurethane as material.

The number of pores was measured using ImageJ in particle analysis mode. The measurement conditions are as follows:

Measuring condition: setting the threshold to 0 to pore recognizable value (normally 60-100)

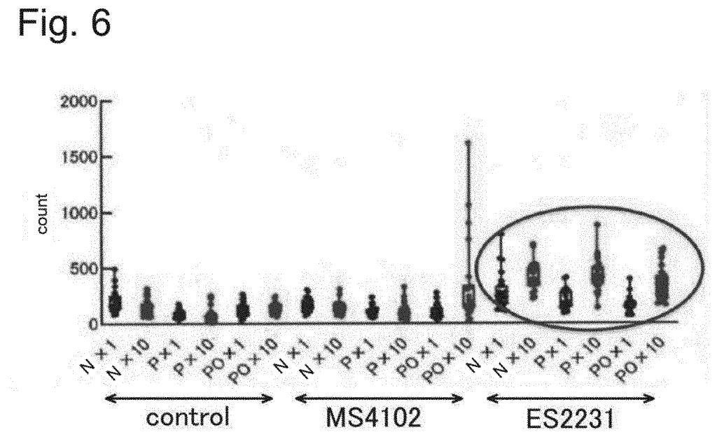

Pore size: 10-∞ pixel (1 μm=7.97 pixel). FIG. 6 shows the result of the measurement. As indicated in the figure, MS4102 strain tends to have a higher increase rate of the number of pores than that for ES2231 strain. It was found from a SEM image observation, which is to be described later, that micron-order bacteria are adsorbed on the surface of the thermoplastic polyurethane, therefore it is considered that degree of bacteria adsorption affected the number of pores.

The meaning of the abbreviations in FIG. 6 are as follows:
N×1: no pretreatment, original metal ion concentrations
N×10: no pretreatment, metal ions 10 times the concentration of the original.
P×1: with plasma pretreatment, original metal ion concentrations
P×10: with plasma pretreatment, metal ions 10 times the concentration of the original
PO×1: with plasma pretreatment and oleic acid treatment, original metal ion concentrations
PO×10: with plasma pretreatment and oleic acid treatment, metal ions 10 times the concentration of the original
Control: no novel decomposing bacteria were made to act (Comparative examples)

SEM Observation of the Surface of Thermoplastic Polyurethane

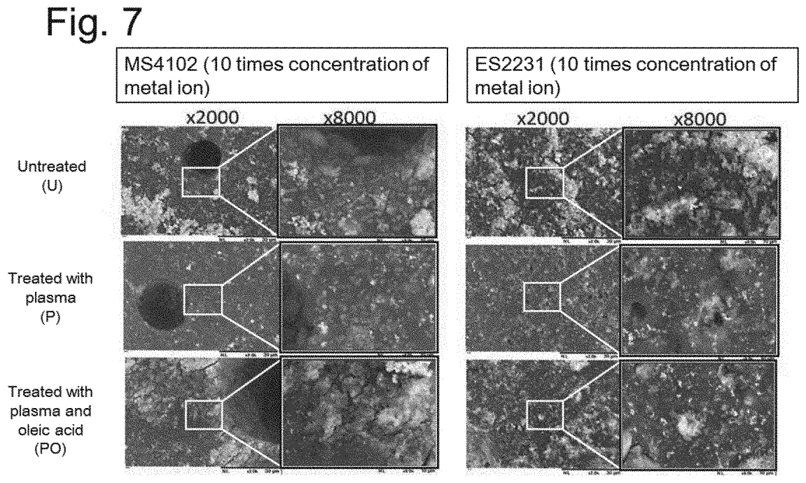

The adsorption of the novel decomposing bacteria of MS4102 strain or ES2231 strain was evaluated by observing 2000× and 8000× photos of SEM image (Miniscope TM3030-HITACHI) of the surface of the thermoplastic polyurethane on which the novel decomposing bacteria were made to act. FIG. 7 shows the result of the SEM observation.

Adsorption of the novel decomposing bacteria was found to be greater for Untreated, P-treated, and PO-treated in that order, which is correlated with the weight reduction ratio that shows the decomposing ability. Oleic acid treatment was found to have a small adsorption, meaning reduction in decomposing ability.
“Untreated” stands for no pretreatment.
“P treated” stands for plasma pretreatment.
“PO treated” stands for plasma- and oleic acid-pretreatment.

Infrared Spectroscopic Analysis

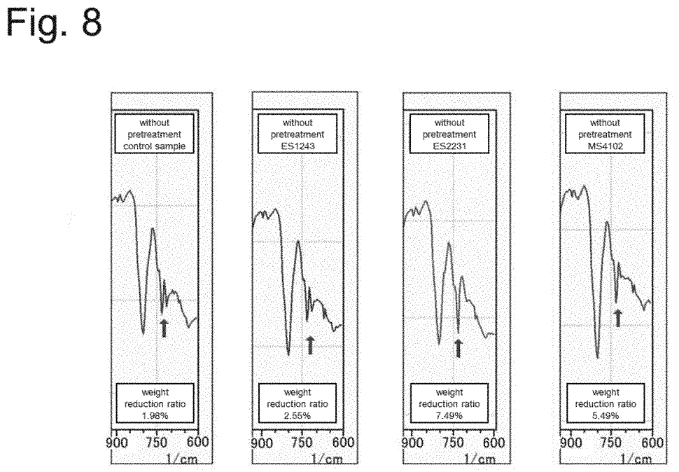

Infrared spectroscopic analysis for the thermoplastic polyurethane on which the novel decomposing bacteria of ES1243 strain, ES2231 stain or MS4102 strain was made to act was performed to compare the peak intensity at 740 cm⁻¹. FIG. 8 shows the result of the analysis. It should be noted that here, no pretreatment was applied to the thermoplastic polyurethane before the novel decomposing bacteria was made to act. Herein, “control” in FIG. 8 stands for the subject on which the novel decomposing bacteria was made to act (comparative examples).

As shown in FIG. 8, the subject without the pretreatment was found to have a double peak (at about 740 and 700 cm⁻¹), which indicates the presence of methylene group (CH2) therein. On the other hand, when the novel decomposing bacteria of ES2231 was made to act, the peak at 700 cm⁻¹ disappears, while the peak ratio at 740 cm⁻¹ has increased. As previously described, the peak at 740 cm⁻¹ of the spectrum in infrared spectroscopic analysis is characteristic of primary amine formed when the urea bond is decomposed, and this presumably led to the disappearance of the double peak of methylene.

As shown in FIG. 8, the weight reduction ratio of thermoplastic polyurethane is correlated with the peak ratio at 740 cm⁻¹ which indicates the decomposition of urea bond. Thus, it was confirmed that the novel decomposing bacteria contribute to urethane decomposition.

As explained above, after the evaluation of urethane decomposing ability of the novel decomposing bacteria, the novel decomposing bacteria are shown to have the highest decomposing ability when the pretreatment step was not conducted prior to the decomposition of the thermoplastic polyurethane. Therefore, the novel decomposing bacteria can decompose thermoplastic polyurethane by a decomposing method that omits the pretreating step, which is expected to lower the cost required in the decomposition process of thermoplastic polyurethane.

Melting Properties

As thermal analysis, A. softening start temperature, B. melting end temperature, C. boiling point, D. decomposition start temperature, E. melting sensitivity (B−A), and F. melting stability (C−B) are evaluated for the melting properties of the material, which allow to determine the advantages of the material when recycled as thermoplastic resin.

Melting sensitivity indicates temperature difference between the start of softening and the end temperature of complete melting, which is an index of how easily the material melts, and the lower the value of melting sensitivity of the material, the greater the melting rate. When using the thermoplastic polyurethane on which the novel decomposing bacteria is made to act is recycled as thermoplastic polyurethane, the higher the melting sensitivity, the more stable the moldability becomes.

Melting stability indicates temperature difference between the melting end temperature and the boiling point of the material, which is an index of the stability of melting state of the material. The higher the melting stability, the higher the stability of melting state of the material, and the material properties of the material as thermoplastic resin improves.

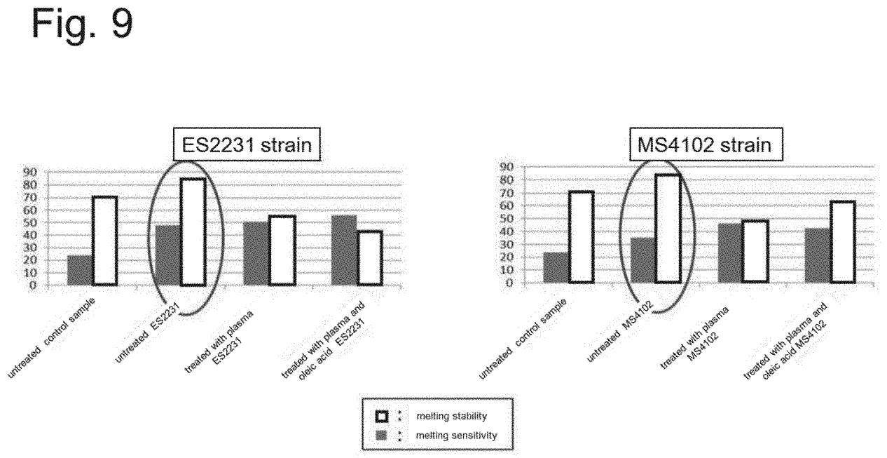

For thermal analysis, Thermogravimetric Differential Thermal Analyzer (TG-DTA) in “Thermo plus EVO2 series” of Rigaku Corporation was employed. Specifically, each sample was heated at a rate of 10° C./min while air was supplied to each sample at a volume of 200 ml/min, and the weight change up to the point at which 700° C. was reached and the thermal changes associated with the physical and chemical changes were detected as a function of temperature compared to the reference material (A1203). The results are shown in Table 5 below. Also, measured melting sensitivity and melting stability are shown in FIG. 9.

TABLE 5

| | | | A | | | D | | |
|---|---|---|---|---|---|---|---|---|
| | | | softening | B | | decomposition | E | F |
| | | | start | melting end | C | start | melting | meling |
| | | | temperature | temperature | boiling point | temperature | sensitivity | stability |
| # | evaluation samples | | (° C.) | (° C.) | (° C.) | (° C.) | (B − A) | (C − B) |
| 1 | untreated | control | 170.4 | 194.3 | 265.6 | 343.3 | 23.9 | 71.3 |
| 2 | | MS4102 | 172.2 | 207.4 | 291.4 | 339.0 | 35.2 | 84.0 |
| 3 | | MS4111 | 169.2 | 214.9 | 270.0 | 335.9 | 45.7 | 55.1 |
| 4 | | ES1243 | 165.8 | 198.1 | 282.0 | 339.9 | 32.3 | 83.9 |
| 5 | | ES2231 | 161.7 | 209.6 | 294.3 | 335.4 | 47.9 | 84.7 |
| 6 | treated | control | 167.7 | 209.2 | 300.6 | 339.1 | 41.5 | 91.4 |
| 7 | with | MS4102 | 174.2 | 220.2 | 268.9 | 336.3 | 46.0 | 48.7 |
| 8 | plasma | MS4111 | 168.6 | 207.3 | 270.0 | 333.8 | 38.7 | 62.7 |
| 9 | | ES1243 | 166.7 | 218.5 | 299.8 | 337.0 | 51.8 | 81.3 |
| 10 | | ES2231 | 170.8 | 221.8 | 276.8 | 337.0 | 51.0 | 55.0 |
| 11 | treated | control | 166.6 | 215.6 | 309.8 | 341.8 | 49.0 | 94.2 |
| 12 | with | MS4102 | 170.7 | 213.0 | 276.5 | 337.2 | 42.3 | 63.5 |
| 13 | plasma | MS4111 | 167.3 | 217.4 | 300.5 | 341.2 | 50.1 | 83.1 |
| 14 | and oleic | ES1243 | 169.3 | 213.5 | 284.9 | 342.4 | 44.2 | 71.4 |
| 15 | acid | ES2231 | 170.1 | 226.3 | 270.3 | 344.1 | 56.2 | 44.0 |

“Untreated” in Table 5 and FIG. 9 indicates that no pretreatment was applied to the thermoplastic polyurethane before the novel decomposing bacteria was made to act. “Treated with plasma” in Table 5 and FIG. 9 indicates that plasma pretreatment was applied to the thermoplastic polyurethane before the novel decomposing bacteria was made to act, and “treated with plasma and oleic acid” in Table 5 and FIG. 9 indicates that plasma pretreatment and oleic acid pretreatment were applied to the thermoplastic polyurethane before the novel decomposing bacteria was made to act “Control” in Table 5 and FIG. 9 indicates that no novel decomposing bacteria were made to act (Comparative examples)

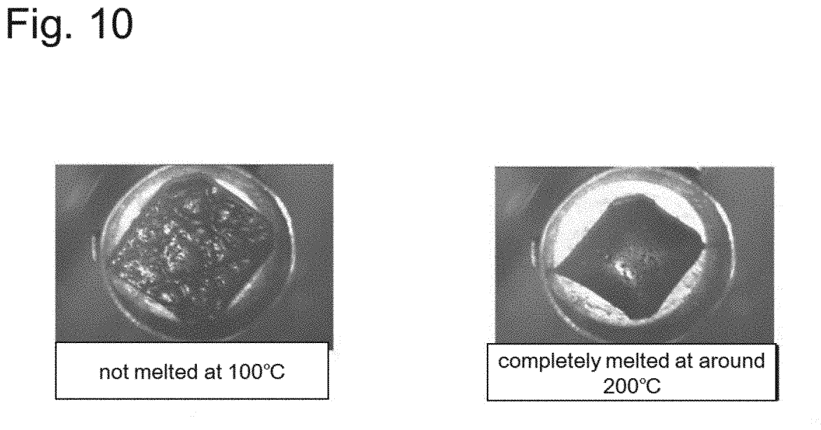

FIG. 10 is a photograph showing a state after the thermoplastic polyurethane, on which MS2231 strain is made to act, is heated.

By decomposing thermoplastic polyurethane with ES2231 strain of the novel decomposing bacteria, the bacteria act on hydrogen bond at the end of the urea bond weakening its bonding strength, and molecular misalignment tends to occur when thermal energy is imparted, which causes the thermoplastic polyurethane to melt completely at 200° C. just as in the case of powder material.

In particular, thermoplastic polyurethane on which the novel decomposing bacteria is made to act without pretreatment has the highest thermal stability, and its material properties changed to that for material-recyclable thermoplastic resin.

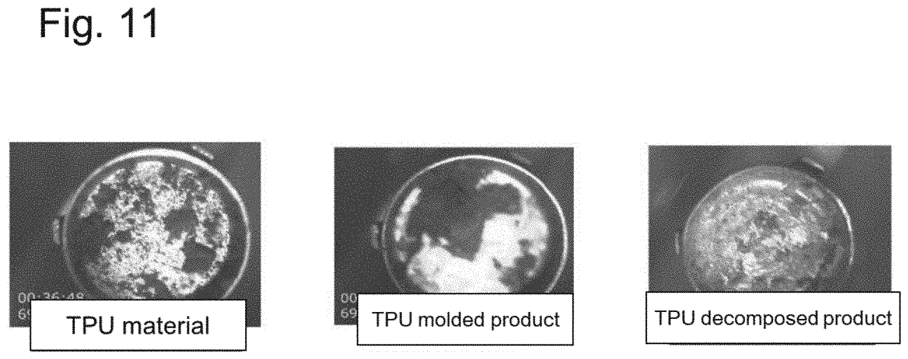

FIG. 11 is a photograph showing the comparison of the amount of ash residues after the thermoplastic polyurethane is heated to 700° C. to completely decompose the resin component. As exhibited in FIG. 11, ash residues and carbonized matter remain for powder material or molded waste material of thermoplastic polyurethane, while thermoplastic polyurethane on which the novel decomposing bacteria was made to act decomposes completely with hardly any residues, proving that it has environmentally friendly material properties.

“TPU material” in FIG. 11 stands for powdery material for the thermoplastic polyurethane. Similarly, “TPU molded product” in FIG. 10 stands for waste material for the molded thermoplastic polyurethane, while “TPU decomposed product” in FIG. 10 stands for the thermoplastic polyurethane after the novel decomposing bacteria of ES2231 was made to act.

Furthermore, as described before, molded product of the thermoplastic polyurethane on which the novel decomposing bacteria was not made to act carbonizes without complete melting as temperature goes up because partial cross-linking when melt-solidifying strengthen the molecular structure, therefore it has not been recycled as thermoplastic resin (see FIG. 12).

As explained above, the selected novel decomposing bacteria was found to have a high decomposing ability against hardly decomposable thermoplastic polyurethane. In particular, ES2231 strain under the condition of no pretreatment of thermoplastic polyurethane shows a high decomposing ability, therefore, a drastic reduction in cost in the process of decomposing thermoplastic polyurethane can be expected.

Conventionally, a firm cross-linking forms in the process of melt-solidifying molded products of thermoplastic polyurethane rendering a uniform melting of them impossible. However, their melting properties as thermoplastic resin changed by treating them with the novel decomposing bacteria.

Melting properties of the molded product of the thermoplastic polyurethane without pretreatment is found to have a highest melting stability, showing a suitable melting property as thermoplastic resin. Thus, recycling processes of thermoplastic polyurethane as thermoplastic resin can be simplified as well.

Furthermore, thermoplastic polyurethane on which the selected novel decomposing bacteria was made to act decomposes completely and produces almost no decomposition residue after heating, having environmentally friendly material properties.

### DESCRIPTION OF THE REFERENCE NUMERALS

141 special structure A (highly agglomerated polyester)
142 special structure B (end group)

1. Microorganisms belonging to genus Pseudomonas and a genus Sinomonas that are capable of decomposing thermoplastic polyurethane containing urea bond.

2. The microorganism according to claim 1, wherein the microorganism is Pseudomonas hibiscicola MS4102 strain specified by Accession No. NITE P-03612.

3. The microorganism according to claim 1, wherein the microorganism is Sinomonas atrocyanea ES2231 strain specified by Accession No. NITE P-03613.

4. A method for decomposing thermoplastic polyurethane that includes a step of making the microorganism according to claim 1 act on the thermoplastic polyurethane containing urea bond.

5. A method for decomposing thermoplastic polyurethane that includes a step of making the microorganism according to claim 2 act on the thermoplastic polyurethane containing urea bond.

6. A method for decomposing thermoplastic polyurethane that includes a step of making the microorganism according to claim 3 act on the thermoplastic polyurethane containing urea bond.

7. A method for selecting microorganisms capable of decomposing thermoplastic polyurethane including
a step of burying in soil a mixed sample of thermoplastic polyurethane containing urea bond and polyurethane not containing urea bond and
a step of collecting the microorganism adsorbing to the mixed sample after confirming the urethane decomposition by analysis of the buried mixed sample.

8. The method for selecting microorganisms capable of decomposing thermoplastic polyurethane according to claim 5, wherein the method includes a step of culturing the collected microorganism in an inorganic salt medium containing 1, 3-dimethylurea, 1, 3-diethylurea or 1, 3-dimethylurea and 1, 3-diethylurea.
